(19) United States
(12) Patent Application Publication
(10) Pub. No.: US 2020/0306314 A1
(43) Pub. Date: Oct. 1, 2020

(54) METHOD FOR TREATING AND/OR PREVENTING ALZHEIMER'S DISEASE

(71) Applicant: Taiwan Mitochondrion Applied Technology Co., Ltd., Zhubei City (TW)

(72) Inventors: Han-Chung CHENG, Zhubei City (TW); Chi-Tang TU, Zhubei City (TW); Chih-Kai HSU, Zhubei City (TW)

(21) Appl. No.: 16/832,081

(22) Filed: Mar. 27, 2020

(30) Foreign Application Priority Data
Mar. 27, 2019 (TW) 108110798

(51) Int. Cl. A61K 35/30; A61P 25/28

(52) U.S. Cl. CPC A61K 35/30; A61K 9/0012; A61P 25/28

(57) ABSTRACT

The present invention is related to a method for treating and/or preventing Alzheimer's disease, especially using mitochondria for treatment and/or prevention of Alzheimer's disease.

### CROSSED-REFERENCE TO RELATED APPLICATION

This application claims priority to and the benefit of Taiwan Patent Application No. 108110798, filed on Mar. 27, 2019, in the Taiwan Intellectual Property Office, the entire content of which is incorporated herein by reference.

### 1. FIELD OF THE INVENTION

The present invention is related to a method for treating and/or preventing Alzheimer's disease, especially using mitochondria for treatment and/or prevention of Alzheimer's disease.

### 2. DESCRIPTION OF THE PRIOR ART

Alzheimer's disease (AD) is the most common type of dementia. As the number of people with Alzheimer's worldwide continues to increase, and there is currently no effective treatment, it is one of the global concerns. There are currently more than 50 million Alzheimer's patients worldwide, and it is expected to increase to more than 130 million by 2050. At present, the cost of medical care for Alzheimer's disease worldwide is about 800 billion U.S. dollars, which is an enormous economic burden on society.

The exact cause of Alzheimer's disease is still unknown, but plaques and neurofibrillary tangles (NFTs) are observed in brain tissue from Alzheimer's disease patients. Plaques are the result of amyloid beta peptide (Aβ) aggregation in the brain. Current research suggests that the aggregation of β-amyloid peptides will lead to an increase in oxidative stress and neurotoxicity in the brain, which cause damage and necrosis of brain nerve cells. Therefore, the abnormal aggregation of β-amyloid peptides is considered to be the main cause of Alzheimer's disease.

Two types of drugs are currently used to relieve Alzheimer's disease, one is cholinesterase inhibitor, the other is N-methyl-D-aspartate (NMDA) receptor antagonist. Acetylcholine is an important neurotransmitter in the brain. Decreased acetylcholine concentration in patients with Alzheimer's disease can cause degeneration of the cerebral cortex and memory loss. Therefore, in patients with mild and moderate Alzheimer's disease, acetylcholinease inhibitors are often used to improve memory loss. Commonly used acetylcholine inhibitors include rivastigmine, donepezil, galantanime. However, the long-term follow-up studies show that the efficacy of these drugs is quite limited, and there are doubts about whether it can actually improve Alzheimer's disease. In addition, active NMDA receptors cause excitatory neurons, but overactivation of NMDA receptors can leads to excitotoxicity, by which nerve cells are damaged or killed. Therefore, NMDA receptor antagonists are used to block activation of NMDA receptors in order to stop excitotoxic neuronal death. NMDA receptor antagonists are also commonly used to treat patients with moderate and severe Alzheimer's disease. Memantine is the only drug of this class. However, in a 2011 study published by Schneider et al., there was no sufficient evidence to suggest that memantine could slow the treatment of Alzheimer's disease. Although many new therapies are now being introduced, such as stem cell therapy or gene therapy, these novel therapies are still in research stage and there are doubts about safety and carcinogenicity of these novel therapies.

### SUMMARY OF THE INVENTION

The first aspect of the present invention provides a method for treating and/or preventing Alzheimer's disease in a subject.

The second aspect of the present invention provides a method for improving memory deficits and/or learning impairments of a subject.

The third aspect of the present invention provides a method for reducing a concentration of β-amyloid peptides in a cell of a subject.

These and other aspects of the present invention will become apparent from the following description of the preferred embodiment taken in conjunction with the following drawings, although variations and modifications therein may be effected without departing from the spirit and scope of the novel concepts of the disclosure.

### BRIEF DESCRIPTION OF THE DRAWINGS

The foregoing summary, as well as the following detailed description of the invention, will be better understood when read in conjunction with the appended drawings. For the purpose of illustrating the present invention, the attached drawings illustrate some, but not all, alternative embodiments. It should be understood, however, that the invention is not limited to the precise arrangements and instrumentalities shown. These figures, which are incorporated into and constitute part of the specification, assist in explaining the principles of the invention.

FIGS. 1A, 1B, and 1C show the cell morphology of neurons derived from induced pluripotent stem cells of patients with down syndrome (DS-Neurons) treated with 0 μg (control), 15 μg/ml, and 40 μg/ml of mitochondria, respectively, for 24 hours. FIGS. 1D, 1E, and 1F show the cell morphology of DS-Neurons treated with 0 μg (control), 15 μg/ml, and 40 μg/ml of mitochondria, respectively, for 48 hours.

FIG. 2A shows concentrations of amyloid beta peptide 40 (Aβ40) in DS-Neurons treated with 0 μg (control), 15 μg/ml, and 40 μg/ml, respectively, for 24 hours. FIG. 2B shows concentrations of amyloid beta peptide 40 (Aβ40) in DS-Neurons treated with 0 μg (control), 15 μg/ml, and 40 μg/ml, respectively, for 48 hours. Data are represented as the mean ±standard error of mean (SEM). In comparison with the control group, ###, p<0.001.

FIG. 3A shows concentrations of amyloid beta peptide 42 (Aβ42) in DS-Neurons treated with 0 μg (control), 15 μg/ml, and 40 μg/ml, respectively, for 24 hours. FIG. 3B shows concentrations of amyloid beta peptide 42 (Aβ42) in DS-Neurons treated with 0 μg (control), 15 μg/ml, and 40 μg/ml, respectively, for 48 hours. Data are represented as the mean±SEM. In comparison with the control group, #, p<0.05; ##, p<0.01.

FIGS. 4A and 4B show the average escape latency and swimming distance of mice with different treatments in water maze tests, respectively. Mice were randomly divided into Group 1 (control), Group 2 (injection with β-amyloid peptide), Group 3 (injection with β-amyloid peptide and 30 μg mitochondria), and Group 4 (injection with β-amyloid peptide and 60 μg mitochondrion). Data are represented as the mean±SEM. In comparison with the control group, #, p<0.05; ##, p<0.01.

FIGS. 5A, 5B, and 5C show the expression levels of Aβ42, β-secretase, and γ-secretase in the brains of mice with different treatments, respectively. Mice were randomly divided into Group 1 (control), Group 2 (injection with β-amyloid peptide), Group 3 (injection with β-amyloid peptide and 30 μg mitochondria), and Group 4 (injection with β-amyloid peptide and 60 μg mitochondrion). Data are represented as the mean±SEM. In comparison with the control group, #, p<0.05; ##, p<0.01; ###, p<0.001.

### DETAILED DESCRIPTION OF THE PREFERRED EMBODIMENT

It should be understood that both the foregoing general description and the following detailed description are exemplary and explanatory but are not restrictive of the invention as claimed. Certain details of one or more embodiments of the invention are set forth in the description below. Other features or advantages of the present invention will be apparent from the non-exhaustive list of representative examples that follows, and also from the appending claims.

The present invention provides a method for treating and/or preventing Alzheimer's disease in a subject, comprising administering to the subject a composition comprising an effective dose of isolated mitochondria. The “Guidance for Industry and Reviewers Estimating the Safe Starting Dose in Clinical Trials for Therapeutics in Adult Healthy Volunteers” published by the U.S. Department of Health and Human Services Food and Drug Administration discloses “a human equivalent dose (HED)” may be obtained by calculations from the following formula: HED=animal dose in mg/kg×(animal weight in kg/human weight in kg)^{0.33}.

In some embodiments, the doses used in the mouse trials are 30 μg per mouse and 60 μg per mouse (a mouse weighs 30 mg), and the HEDs for a 60 kg human calculated are 0.0815 and 0.163 mg/kg, respectively.

In some embodiments, the mitochondria are administered to a subject, preferably a human, and the dosage is at least about 0.07 mg/kg, about 0.08 mg/kg, about 0.09 mg/kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.20 mg/kg, about 0.21 mg/kg, about 0.22 mg/kg, about 0.23 mg/kg, about 0.24 mg/kg, or about 0.25 mg/kg body weight.

In some embodiments, the isolated mitochondria are isolated exogenous mitochondria.

In some embodiments, the isolated mitochondria are derived from a stem cell. In some preferred embodiments, the isolated mitochondrial are derived from an adipose stem cell.

In some embodiments, the isolated mitochondria improve memory deficits and/or learning impairments caused by Alzheimer's disease.

In some embodiments, the composition comprises the isolated mitochondria and an aqueous buffer, and the isolated mitochondria are not encapsulated.

In some embodiments, the composition is administered to a subject by brain injection.

The invention further provides a method for improving memory deficits and/or learning impairments of a subject, comprising administering to the subject a composition consisting essentially of an effective dose of isolated mitochondria.

The invention also provides a method for reducing a concentration of β-amyloid peptides in a cell of a subject, comprising administering to the subject a composition consisting essentially of an effective dose of isolated mitochondria.

The invention further provides a pharmaceutical composition for treating and/or preventing Alzheimer's disease, comprising an effective dose of isolated mitochondria and a pharmaceutically acceptable carrier.

Unless otherwise defined, all technical and scientific terms used herein have the same meaning as commonly understood by one of ordinary skill in the art to which this invention pertains. In the case of conflict, the present document, including definitions will control.

As used herein, the term “isolated mitochondria” refers to the mitochondria that are separated from a cell in which they were originally present and maintained in a non-cellular environment that maintains their activity. In some embodiments, the isolated mitochondria of the present invention are in a buffer solution capable of maintaining the activity of the mitochondria. In some embodiments, the buffer solution contains a protease inhibitor.

As used herein, the term “isolated mitochondria are not encapsulated” refers to that the mitochondria that are separated from a cell in which they were originally present and maintained in a non-cellular environment that maintains their activity are not packaged by any drug delivery system. Drug delivery systems include, but are not limited to liposomes, lipid bilayer, lipid micelles, lipid raft, clathrin-coated vesicles.

As used herein, the term “aqueous buffer” or “buffer solution”, which are used interchangeably, refers to an aqueous solution consisting of a mixture of a weak acid and its conjugate base, or a weak alkali and its conjugate base, which can reduce the effect of addition of a strong acid or a strong alkali on the pH of the solution to a certain extent, and therefore maintain the stability of the pH of the solution. In some embodiments, the buffer solution includes sucrose, ethylene glycol tetraacetic acid (EGTA), and N-(2-Hydroxyethyl) piperazine-N′-2-ethanesulfonic Acid (HEPES). In some embodiments, the buffer solution is SHE buffer, including 0.25 M sucrose, 0.5 mM EGTA, and 3 mM HEPES.

As used herein, the term “exogenous mitochondria” or “foreign mitochondria” refers to mitochondria that do not originate from the subject to whom the mitochondria are administered. Thus, “administering exogenous mitochondria to a subject” means administering to the subject mitochondria that are not from the subject themselves. Sources of exogenous mitochondria include, but are not limited to, another individual of the same species and an individual of different species (heterogeneous). For example, a human subject is administered with exogenous mitochondria, which may originate from a human who is not the subject, a group of humans not including the subject, or other species, such as mice, rats, rabbits, cattle, sheep, horses, monkeys, apes.

The methods provided by the present invention reduce the concentration of β-amyloid peptides in a cell of a subject by administering to the subject an effective dose of isolated mitochondria, thereby treating and/or preventing azimuth Hemmer's disease, and/or improving in memory deficits and/or learning impairments. β-amyloid peptides are produced through the proteolytic processing of a transmembrane protein, amyloid precursor protein (APP), by β-secretase, also known as β-site amyloid precursor-cleaving enzyme (BACE), and γ-secretase. Therefore, when the activities of β-secretase and γ-secretase in a tissue decrease, the concentration of β-amyloid peptides in the tissue reduces as well. β-amyloid peptides mainly exist in two forms, β-amyloid peptide 40 (Aβ40, with 40 amino acids) and β-amyloid peptide 42 (Aβ42, with 42 amino acids). Aβ42 is not easily broken down and easily aggregates outside nerve cells to form plaques. The β-amyloid peptide content in blood is a common indicator of early stage of Alzheimer's disease. Therefore, the content of Aβ40 and Aβ42 and the content of β-secretase and γ-secretase are often used as the basis and indicators for studying the formation of Alzheimer's disease and the efficacy of a drug.

As used herein, the term “reducing a concentration of β-amyloid peptides” refers to that the concentration of β-amyloid peptides in the brain of a subject treated with the mitochondria provided by the present invention is lower than the concentration of β-amyloid peptides in the brain of a subject not treated with the mitochondria provided by the present invention. Alternatively, the term refers to that the concentration of β-amyloid peptides in the brain of a subject after the subject was treated with the mitochondria provided by the present invention is lower than the concentration of β-amyloid peptides in the brain of the same subject before the subject was treated with the mitochondria provided by the present invention. In some embodiments, the term “reducing a concentration of β-amyloid peptides” refers to that a subject who was induced to have an abnormally high concentration of β-amyloid peptides has a reduced concentration of β-amyloid peptides after the subject was treated with isolated mitochondria, compared to another subject who had the same induction to have an abnormally high concentration of β-amyloid peptides but was not treated with isolated mitochondria.

As used herein, the term “neurons derived from induced pluripotent stem cells of patients with Down Syndrome (DS-Neurons)” refers to the neurons derived from Down Syndrome induced pluripotent stem cells (DS-iPSCs), which are induced from somatic cells of Down Syndrome (DS) patients. Down Syndrome is a genetic disorder caused by the presence of all or part of a third copy of chromosome 21. In humans, the gene for amyloid precursor protein (APP) is located on chromosome 21. Therefore, most DS patients have early onset major neurocognitive disorder and early onset Alzheimer's disease, and β-amyloid accumulation can be found in their brains, which make DS patients a research model for Alzheimer's disease. Down Syndrome induced pluripotent stem cells (DS-iPSCs), which are derived from somatic cells of Down Syndrome (DS) patients, have the ability to differentiate into neurons (DS-Neurons). DS-Neurons produce Aβ40and Aβ42, which are important indicators of Alzheimer's disease, and induce aggregation of Aβ and formation of plaques. Therefore, DS-Neurons can be used as a drug screening platform, in which the clearance of Aβ is an important indicator of a potential drug for treating and/or delaying Alzheimer's disease. In some embodiments, the mitochondria provided by the present invention can effectively reduce β-amyloid aggregation in DS-Neurons and can be used for treating and/or delaying Alzheimer's disease.

As used herein, the term “pharmaceutical composition” refers to any formulation wherein the mitochondria of the invention may be formulated, stored, preserved, altered, administered, or a combination thereof. As described below, the formulation may comprise any pharmaceutically-acceptable diluent, adjuvant, buffer, excipient, carrier, or combination thereof. In general, components of the formulation are selected on the basis of the mode and route of administration, and standard pharmaceutical practice.

As used herein, the term “pharmaceutically acceptable carrier” means that any substance or combination thereof with the mitochondria of the present invention can be physically or chemically mixed, dissolved, suspended, or otherwise combined to yield the pharmaceutical composition of the present invention.

As used herein, the term “pharmaceutically effective amount” refers to an amount capable of or sufficient to maintain or produce a desired physiological result, including but not limited to treating, reducing, eliminating, substantially preventing, or prophylaxing, or a combination thereof, a disease, disorder, or combination thereof. A pharmaceutically effective amount may comprise one or more doses administered sequentially or simultaneously. Those skilled in the art will know to adjust doses of the present invention to account for various types of formulations, including but not limited to slow-release formulation.

As used herein, the terms “treat,” “treating,” “treatment” and “improve,” “improving,” “improvement”, which are used interchangeably, refer to being able to cure, reduce, stop the progression, slow down the progression, advantageously change, eliminate, or a combination thereof, any aspect of a disease, condition, or combination thereof.

As used herein, the term “prevent,” “preventing,” or “prevention” refers to being able to substantially preclude, avert, obviate, forestall, stop, hinder, or a combination thereof, any aspect of a disease, condition, or combination thereof from happening, especially by advance action.

As used herein, the term “subject” refers to any individual to whom administration of the present invention is directed. A subject may be, for example, a mammal. The subject may be a human or veterinary animal, without regard to sex, age, or any combination thereof, and including fetuses. A subject may optionally be afflicted with, at risk for, or a combination thereof a particular disease, disorder, or combination thereof.

As used herein, the articles “a,” “an,” and “any” refer to one or more than one (i.e., at least one) of the grammatical object of the article. For example, “an element” means one element or more than one element.

As used herein, “around,” “about,” or “approximately” shall generally mean within 20 percent, preferably within 10 percent, and more preferably within 5 percent of a given value or range. Numerical quantities given herein are approximate, meaning that the term “around,” “about,” or “approximately” can be inferred if not expressly stated.

The present invention is further illustrated by the following examples, which are provided for the purpose of demonstration rather than limitation. Those skilled in the art should, in light of the present disclosure, appreciate that many changes can be made in the specific embodiments which are disclosed and still obtain a like or similar result without departing from the spirit and scope of the invention.

### EXAMPLE

### Example 1

### Preparation of Neurons Derived from Induced Pluripotent Stem Cells of Patients with Down Syndrome (DS-Neurons)

1. Cell Culture of Induced Pluripotent Stem Cells of Patients with Down Syndrome (DS-iPSCs)

DS-iPSCs (provided by Dr. Honglin Su, Department of Life Sciences, National Chung Hsing University, Taichung, Taiwan) were cultured in a serum-free medium, Essential 8™ (Life Technology, USA) and subjected to attachment culture with Matrigel™ (Becton-Dickinson, USA). Then, the culture medium was removed, and the cells were washed twice with Dulbecco's phosphate-buffered saline (DPBS, Corning, Cat. no. 21-031-CV, USA). After that, Accutase™ (Merck Millipore, USA) was added to the cells and reacted with the cells at 37° C. for 2 to 5 minutes, and then the medium was added to stop the reaction. The cells were washed, dispersed, and centrifuged at 1,000 rpm for 2 minutes to remove the supernatant. The cells were then subcultured on a culture plate containing fresh serum-free medium, Essential 8™, for about 3 to 5 days, and the culture medium was changed every day.

2. Differentiation of Induced Pluripotent Stem Cells of Patients with Down Syndrome (DS-iPSCs) into Neurons

The above-mentioned DS-iPSCs were cultured to 80 to 90% confluency, and the cells were washed twice with DPBS. After that, Accutase™ (Merck Millipore, USA) was added to the cells and reacted with the cells at 37° C. for 2 to 5 minutes, and then the medium was added to stop the reaction. The cells were washed, dispersed, and centrifuged at 800 rpm for about 2 minutes to remove the supernatant. The cells were then cultured in Dulbecco's Modified Eagle Medium: Nutrient Mixture F-12 (DMEM-F12) supplemented with 20% (v/v) knock out serum replacement (KSR, Life Technology, USA) for 2 days to obtain suspension of embryonic bodies.

The suspended embryoid bodies were placed in a centrifuge tube, and after embryoid bodies precipitated naturally, the supernatant was removed. A neural induction medium [each 500 mL of the neural induction medium contains 326 mL of DMEM medium (Life Technologies, Cat. No. 11965-092, USA), 163 mL of F12 medium (Life Technologies, Cat. No. 11765-054, USA), 5 mL N-2 Supplement (Life Technologies, Cat. No. 17502-048, USA), 5 mL of non-essential amino acid (Life Technologies, Cat. No. 11140-035, USA), and 1 mL of heparin (1 mg/mL)] containing small-molecule drugs, BiSF [0.5 μM BIO (Sigma-Aldrich, USA), 10 ng/ml fibroblast growth factor-2 (FGF-2, Peprotech, USA), and 10 μM SB431542 (Sigma-Aldrich, USA)], was added to the cells for suspension culture for 2 days. Then, the suspended embryoid bodies exhibited a circular epithelial cell structure.

After the embryoid bodies precipitated, the supernatant was removed. A neural basal medium [each 521 mL of the neural induction medium contains 500 mL of neural basal medium (Life Technologies, Cat. No. 21103-049, USA), 5 mL of N-2 supplement (Life Technologies, Cat. No. 17502-048, USA), 5 mL of non-essential amino acid (Life Technologies, Cat. No. 11140-035, USA), 1 mL of Heparin (1 mg/mL), and 10 mL of B-27™ Supplement (Life Technologies, Cat. No. 17504-044, USA)] supplemented with 10 ng/ml fibroblast growth factor-2 (FGF-2) was added to the cells for suspension culture for 2 days. After the embryoid bodies precipitated, the embryoid bodies were dispersed with force or Accutase™, seeded on a culture dish coated with 1% (v/v) Matrigel [dissolved in DMEM/F12 medium (Gibco, Cat. No. 11330032, USA)] for cell attachment and culture. After about 2 to 7 days, the embryoid bodies differentiated into neurons, that is, neurons derived from induced pluripotent stem cells of patients with Down Syndrome (DS-Neurons).

3. Subculture of Neurons Derived from Induced Pluripotent Stem Cells of Patients with Down Syndrome (DS-Neurons)

The above-mentioned DS-Neurons were cultured to 80 to 90% confluency, and the cells were washed twice with DPBS. After that, Accutase™ was added to the cells and reacted with the cells at 37° C. for 2 to 5 minutes, and then Neurobasal™ medium (Gibco, Cat. No. 21103049, USA) was added to stop the reaction. The cells were washed, dispersed, and centrifuged at 1,000 rpm for about 2 minutes to remove the supernatant. Neurobasal™ medium was added to adjust the cell concentration to 1×10⁵ cells/mL, and the ROCK inhibitor Y-27632 (with a final concentration of 10 μM) (Merck Millipore Corporation, Cat. No. SCM075, USA) was added. The cells were then seeded on a culture dish coated with 1% (v/v) Matrigel™ (dissolved in DMEM/F12 medium), and Neurobasal™ medium was added for cell culture. On the next day, the Neurobasal™ medium was removed and replaced with Neurobasal™ medium containing 2% (v/v) B-27™ supplement and 10 ng/ml recombinant human Fibroblast Growth Factor (rh-FGF, Gibco, USA). Fresh culture medium was replaced every 2 to 3 days until the next passage.

### Example 2

### Preparation of Mitochondria

The mitochondria of this Example were isolated and purified from human adipose-derived stem cells (ADSCs). Human adipose-derived stem cells were cultured to 1×10⁸ cells, rinsed with DPBS, and reacted with Accutase™ (Merck Millipore, USA) at 37° C. for 2 to 5 minutes. Then, the culture solution was added to stop the reaction, and the cells were washed, dispersed, and centrifuged at 1,000 rpm for about 2 minutes to remove the supernatant. The cells were resuspended with 2 ml of SHE buffer [0.25 M sucrose, 0.5 mM ethylene glycol tetraacetic acid (EGTA), 3 mM N-(2-hydroxyethyl) piperazine-N′-ethanesulfonic acid (HEPES), pH 7.2; all were purchased from Sigma-Aldrich] and then homogenized on ice 15 times with a homogenizer. After that, the cells were centrifuged at 1,000×g for 15 minutes, and the supernatant was transferred to another tube and centrifuged at 9,000×g for 10 minutes. Then, the supernatant was removed, and the pellet (mitochondria) was resuspended with 50 μl SHE buffer containing a protease inhibitor and stored at 4° C. for further use.

### Example 3

### Cell Experiments

1. Cell Treatment

The DS-Neurons obtained in Example 1 were seeded into a 24-well cell culture plate at a density of 2×10⁵ cells/well and cultured in Neurobasal™ medium containing 2% (v/v) B-27™ supplement for 16 to 24 hours. The mitochondria obtained in Example 2 were added to the culture medium at a concentration of 15 μg/ml or 40 μg/ml per well, and then the plate was centrifuged at 1,500×g at 4° C. for 15 minutes to keep the mitochondria at the bottom of the plate. The DS-Neurons were co-cultured with the mitochondria in a 37° C. incubator for 24 or 48 hours. After that, cell morphology was observed under a microscope, and the culture medium in each well was collected. The collected cell culture was then centrifuged at 1,000 rpm for 5 minutes, and the supernatant was stored at −80° C. for further analysis.

2. Enzyme-Linked Immunoadsorbent Assay (ELISA)

The expression levels of Aβ40and Aβ42 of the DS-Neurons were analyzed by ELISA. LEGEND MAX™ β-Amyloid x-40 ELISA Kit (BioLegend, USA) and LEGEND MAX™ β-Amyloid x-42 ELISA Kit (BioLegend, USA) were performed according to manufacturers' instructions. The supernatant obtained from the co-culture of the DS-Neurons and the isolated mitochondria mentioned above was diluted with incubation buffer (BioLegend, USA) at a ratio of 1:5. Fifty (50) μl of the diluted supernatant was added to the reaction plate provided by LEGEND MAX™ β-Amyloid x-40 ELISA Kit and/or LEGEND MAX™ β-Amyloid x-42 ELISA Kit. Each sample was repeat three times. Fifty (50) μl of horseradish peroxidase (HRP) labeled detection antibody (HRP detection antibody, BioLegend, USA) was added to the plates, and the plate was incubated at 4° C. for 16 hours. The mixture was removed, and the plate was washed with 300 μl wash buffer (BioLegend, USA) 5 times. After that, 200 μl 3,3′,5,5′-tetramethylbenzidine (TMB substrate, BioLegend, USA) was added to the plate, and the plate was incubated at room temperature in the dark for 40 to 50 minutes. Then, the absorbance of the sample at 620 nm was measured with an ELISA reader, and the concentration of Aβ40and Aβ42 in a sample was obtained by converting the standard curve of the standard into the absorbance of the sample.

3. Statistical Analysis

For all statistical analyses and comparisons of multiple groups, a GraphPad Prism Program (GraphPad, San Diego, Calif., USA) using ANOVA followed by Tukey's post hoc test has been used. A p<0.05 value was considered significant.

4. Results

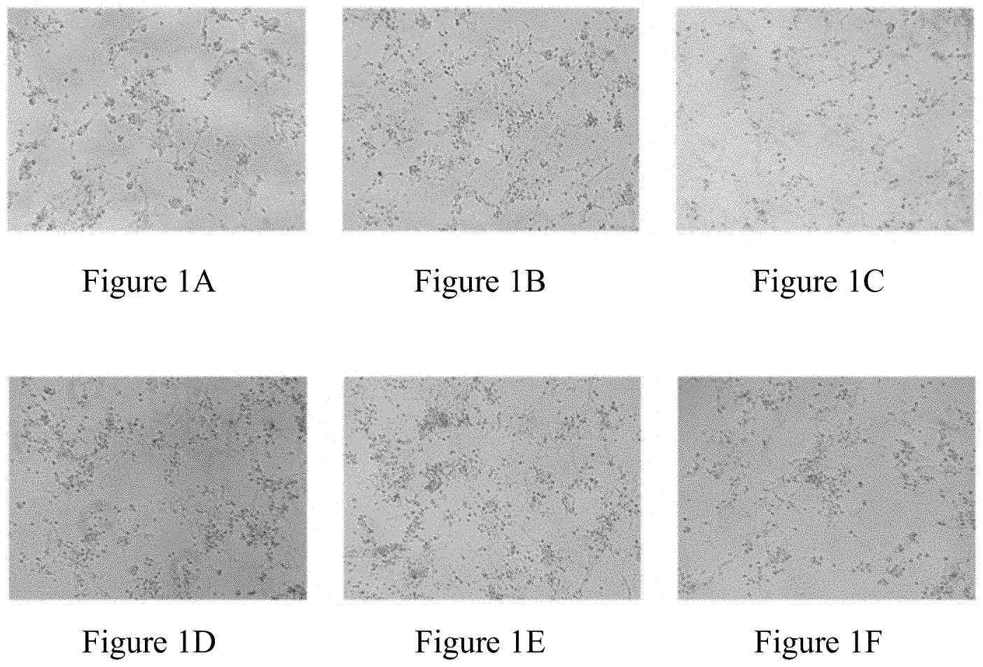

FIGS. 1B, 1C, show the cell morphology of DS-Neurons treated with 15 μg/ml, and 40 μg/ml of mitochondria, respectively, for 24 hours. FIGS. 1E, 1F, show the cell morphology of DS-Neurons treated with 15 μg/ml, and 40 μg/ml of mitochondria, respectively, for 48 hours. The cell morphology of DS-Neurons without treatment with mitochondria (negative controls; FIGS. 1A and 1D) and the cell morphology of DS-Neurons treated with mitochondria (FIGS. 1B, 1C, 1E, and 1F) have no difference. The results indicate that 15 μg/ml or 40 μg/ml of mitochondria cause no cytotoxicity to DS-Neurons.

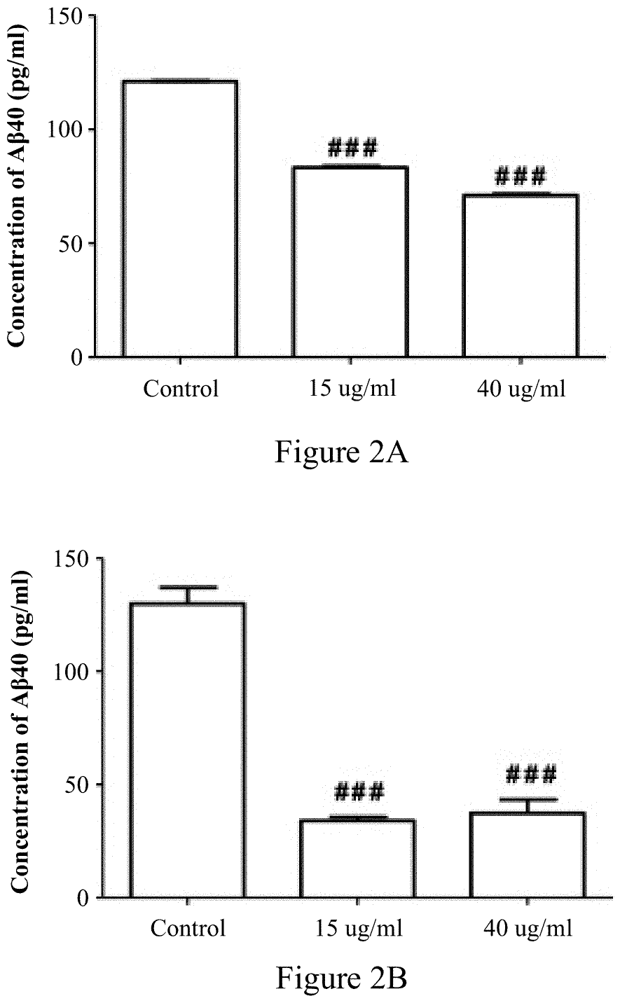

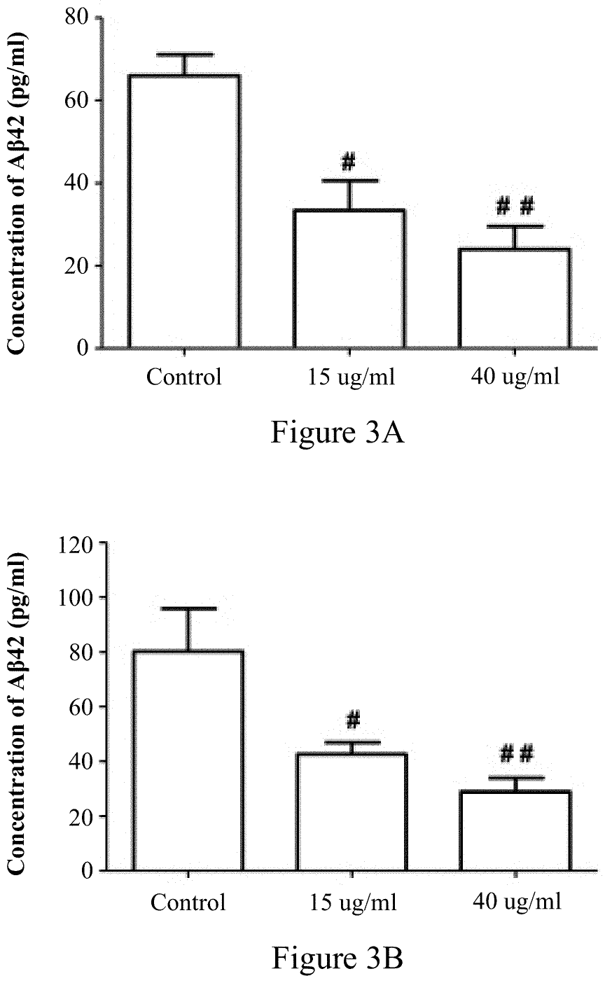

As shown in FIGS. 2A and 2B, DS-Neurons treated with 15 μg/ml or 40 μg/ml for either 24 hours or 48 hours all have significantly lower concentrations of Aβ40 than DS-Neurons without treatment of mitochondria (control) (p<0.001). In addition, as shown in FIGS. 3A and 3B, DS-Neurons treated with 15 μg/ml or 40 μg/ml for either 24 hours or 48 hours all have significantly lower concentrations of Aβ42 than DS-Neurons without treatment of mitochondria (control) (p<0.05 or p<0.01). The results indicate that isolated mitochondria reduce the expression of β-amyloid peptides and can be further used in the treatment of Alzheimer's disease.

### Example 4

### Animal Trials

1. Treatment of Experimental Animals

Twenty-four (24) male C57BL/6 mice (9-week old/25-30 g/mouse, purchased from BioLASCO Co., Ltd, Taiwan), which had never used in any experiments, were randomly divided into 4 groups, and 6 mice for each group. The mice in each group are treated respectively as follows.
Group 1: Each mouse was injected with phosphate buffer solution (PBS) into its lateral ventricle for 14 consecutive days; on the 15th day of the trail, each mouse was injected with SHE buffer into its lateral ventricle (control group, normal mice);
Group 2: Each mouse was injected with 300 pmol Aβ42 into its lateral ventricle every day for 14 consecutive days to cause memory deficits and/or learning impairments; on the 15th day of the trail, each mouse was injected with SHE buffer into its lateral ventricle (Aβ42-treated group, mice with Alzheimer's disease symptoms);
Group 3: Each mouse was injected with 300 pmol Aβ42 into its lateral ventricle every day for 14 consecutive days to cause memory deficits and/or learning impairments; on the 15th day of the trail, each mouse was injected with 30 μg of the isolated mitochondria obtained in Example 2 (Aβ42+mito 30-treated group, mice with Alzheimer's disease symptoms treated with 30 μg isolated mitochondria); and
Group 4: Each mouse was injected with 300 pmol Aβ42 into its lateral ventricle every day for 14 consecutive days to cause memory deficits and/or learning impairments; on the 15th day of the trail, each mouse was injected with 60 μg of the isolated mitochondria obtained in Example 2 (Aβ42+mito 60-treated group, mice with Alzheimer's disease symptoms treated with 60 μg isolated mitochondria).
On the 27th day of the trial, all of the mice were used for behavior experiments (water maze learning).

2. Water Maze Learning

A plastic circular pool 183 cm in diameter was filled with water (25±2° C.). A circular platform was placed at a specific location from the edge of the pool and submerged below the water surface, for mice to stand and take a rest. Water was made cloudy by adding toxic-free dye. Mice have to learn by recognizing and memorizing distinctive visual cues set on the wall of the pool. For memory and spatial learning, mice were subjected to 3 trials per day, with one trial early in the morning, one trial at noon, and another in the late afternoon. The training procedure lasted 4 days, and a total of 12 trials were given. The mice were positioned at different starting points spaced equally around the perimeter of the pool in random order. They had 60 seconds to swim in the pool. If a mouse could not find the platform, it was guided to the platform and was allowed to remain there for 20 seconds. The time each mouse took to reach the platform was recorded as the escape latency. The distance each mouse swam to reach the platform was also recorded.

3. Biochemical Analysis

The mice in each group were sacrificed after the behavior experiments, and the expression levels of Aβ42, β-secretase, and γ-secretase were measured. The concentrations of Aβ42 were analyzed by ELISA as described in Example 3. The expression levels of β-secretase and γ-secretase were analyzed by the following methods.

Analysis of expression level of β-secretase. Fifty (50) μl of each sample protein (at a concentration between 25-200 μg/ml) and each serially diluted standard sample were added to 96-well plates. Forty-eight (48) μl of 2× reaction buffer and 2 μl of β-secretase substrate were added to each sample. The mixture was incubated at 37° C. in dark for 1 to 2 hours. The expression levels of sample proteins were measure using a plate reader with an excitation wavelength of 335 to 355 nm and an emission wavelength of 495 to 510 nm. The expression level (relative fluorescence unit, RFU) of β-secretase in a sample was obtained by converting the standard curve of the serially diluted standard samples.

Analysis of expression level of γ-secretase. One hundred (100) μl of each sample protein and each serially diluted standard sample were added to 96-well plates and incubated at 37° C. for 2 hours. After that, samples were removed, 100 μl of biotin-antibody was added to each well and incubated at 37° C. for 1 hour. Then, the antibody was removed, each well was rinsed with wash buffer 5 times, and 90 μl of 3,3′,5,5′-Tetramethylbenzidine (TMB) substrate was added to each well and incubated at 37° C. in dark for 15 to 30 minutes. Finally, 50 μl of stop solution was added to each well, and the absorbance of a sample at 450 nm was measured with a fluorescence reader. The expression level (RFU) of γ-secretase in a sample was obtained by converting the standard curve of the serially diluted standard samples.

4. Statistical Analysis

For all statistical analyses and comparisons of multiple groups, a GraphPad Prism Program (GraphPad, San Diego, Calif., USA) using ANOVA followed by Tukey's post hoc test has been used. A p<0.05 value was considered significant.

5. Results

5.1 Isolated Mitochondria Improve Spatial Learning Impairments and Memory Deficits Caused by β-Amyloid Peptides.

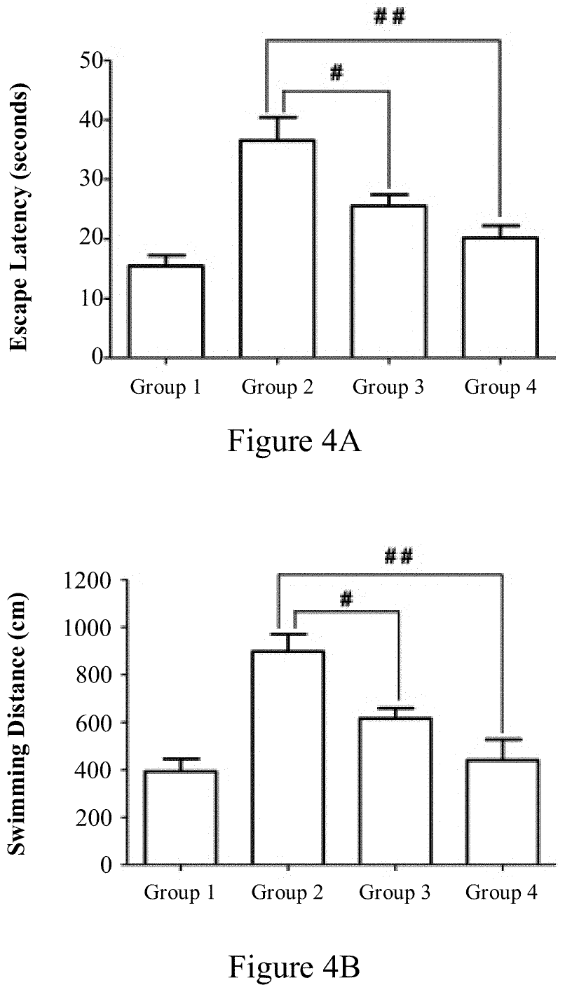

In this Example, animal model with learning impairments and memory deficits caused by β-amyloid peptides was used to study the effect of isolated mitochondria on Alzheimer's disease. As shown in FIGS. 4A and 4B, mice which were injected with Aβ42 only (Group 2) spend the longest time and swam the longest distance to reach the platform. FIG. 4A also shows that mice which were injected with Aβ42 and followed by treatment of 30 μg or 60 μg of the isolated mitochondria (Group 3 or Group 4) had significantly less escape latency than mice of Group 2 (p<0.05 or p<0.01), and the isolated mitochondria improved spatial learning and memory of mice in a dose-dependent fashion. In addition, FIG. 4B shows that mice which were injected with Aβ42 and followed by treatment of 30 μg or 60 μg of the isolated mitochondria (Group 3 or Group 4) swam significantly shorter distance than mice of Group 2 (p<0.05 or p<0.01). The results indicates that the isolated mitochondria improved spatial learning impairments and memory deficits of mice caused by β-amyloid peptides in a dose-dependent fashion.

5.2 Administration of Isolated Mitochondria Significantly Reduce the Expression Level of Aβ42 and γ-Secretase in the Brain of Mice.

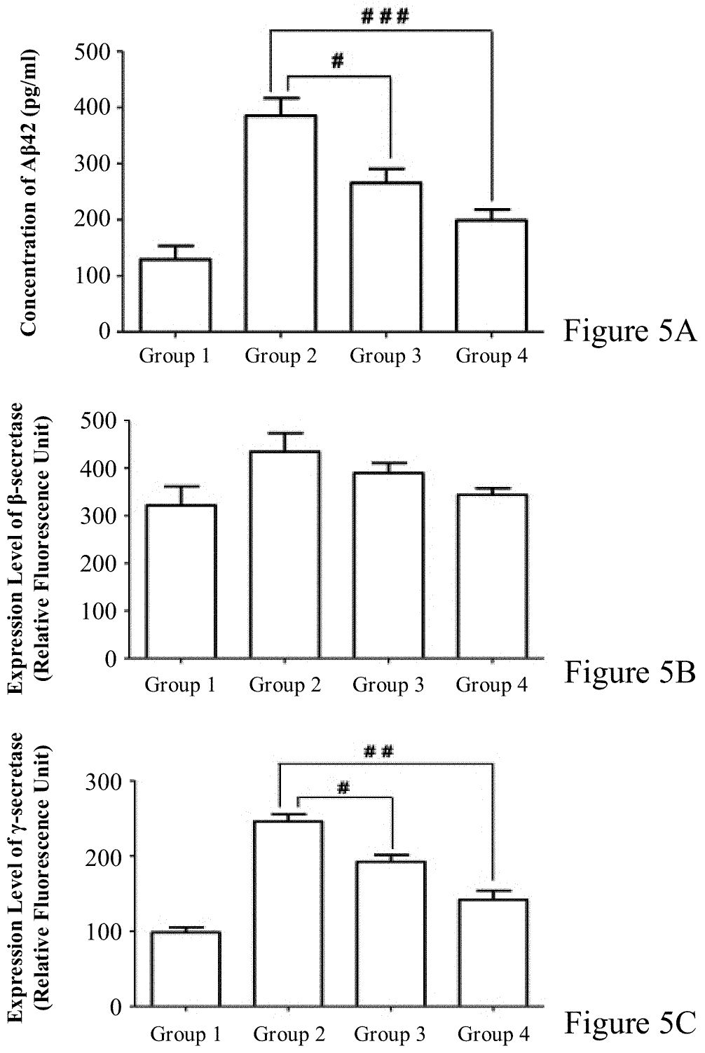

The effects of isolated mitochondria on the expression levels of Aβ42, β-secretase, and γ-secretase in the brains of mice were further analyzed in this Example. As shown in FIGS. 5A, 5B, and 5C, mice which were injected with Aβ42 only (Group 2) had the highest expression levels of Aβ42, β-secretase, and γ-secretase. FIGS. 5A and 5C also show that mice which were injected with Aβ42 and followed by treatment of 30 μg or 60 μg of the isolated mitochondria (Group 3 or Group 4) had significantly less expression levels of Aβ42 and γ-secretase than mice of Group 2 (p<0.05, p<0.01, or p<0.001), and the isolated mitochondria reduce the expression level of Aβ42 and γ-secretase in the brain of mice in a dose-dependent fashion.

Because β-secretase and γ-secretase play important roles in the formation of β-amyloid peptides, and the aggregation of β-amyloid peptides is considered to be the main cause of Alzheimer's disease, the above results indicate that isolated mitochondria reduce the formation and aggregation of β-amyloid peptides in the brains of mice, and further improve the symptoms (memory deficits and learning impairments) of Alzheimer's disease in a dose-dependent fashion.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A method for treating and/or preventing Alzheimer's disease in a subject, comprising administering to the subject a composition consisting essentially of an effective dose of isolated mitochondria.

2. The method of claim 1, wherein the isolated mitochondria is derived from a stem cell.

3. The method of claim 1, wherein the effective dose of isolated mitochondria is at least 0.07 mg/kg of body weight of the subject.

4. The method of claim 1, wherein the composition further comprises an aqueous buffer.

5. The method of claim 4, wherein the aqueous buffer comprises sucrose, ethylene glycol tetraacetic acid (EGTA), and N-(2-hydroxyethyl)piperazine-N′-ethanesulfonic acid (HEPES).

6. The method of claim 1, wherein the composition is administered to the subject by brain injection.

7. A method for improving memory deficits and/or learning impairments of a subject, comprising administering to the subject a composition consisting essentially of an effective dose of isolated mitochondria.

8. The method of claim 7, wherein the isolated mitochondria is derived from a stem cell.

9. The method of claim 7, wherein the effective dose of isolated mitochondria is at least 0.07 mg/kg of body weight of the subject.

10. The method of claim 7, wherein the composition further comprises an aqueous buffer.

11. The method of claim 10, wherein the aqueous buffer comprises sucrose, ethylene glycol tetraacetic acid (EGTA), and N-(2-hydroxyethyl)piperazine-N′-ethanesulfonic acid (HEPES).

12. The method of claim 7, wherein the composition is administered to the subject by brain injection.

13. The method of claim 7, wherein the memory deficits and learning impairments are caused by accumulation of β-amyloid peptides in the brain of the subject.

14. A method for reducing a concentration of β-amyloid peptides in a cell of a subject, comprising administering to the subject a composition consisting essentially of an effective dose of isolated mitochondria.

15. The method of claim 14, wherein the isolated mitochondria is derived from a stem cell.

16. The method of claim 14, wherein the effective dose of isolated mitochondria is at least 0.07 mg/kg of body weight of the subject.

17. The method of claim 14, wherein the composition further comprises an aqueous buffer.

18. The method of claim 17, wherein the aqueous buffer comprises sucrose, ethylene glycol tetraacetic acid (EGTA), and N-(2-hydroxyethyl)piperazine-N′-ethanesulfonic acid (HEPES).

19. The method of claim 14, wherein the composition is administered to the subject by brain injection.

20. The method of claim 14, wherein the cell is a brain neural cell.
